(19) United States
(12) Patent Application Publication
(10) Pub. No.: US 2025/0248839 A1
(43) Pub. Date: Aug. 7, 2025

(54) URINARY CATHETER DRAINAGE BAG COUPLING DEVICE

(71) Applicant: Ohio State Innovation Foundation, Columbus, OH (US)

(72) Inventors: Ryan BRUNE, Columbus, OH (US); Karen MEADE, Delaware, OH (US); Bridgette KREUZ, Columbus, OH (US); Barbara POTTS, Alexandria, OH (US); Lindsay RING, Columbus, OH (US); Michelle KUHN, Columbus, OH (US); Garrett CARDER, Columbus, OH (US)

(21) Appl. No.: 18/856,616

(22) PCT Filed: Apr. 13, 2023
(86) PCT No.: PCT/US23/18532

§ 371 Date: Oct. 13, 2024

Related U.S. Application Data
(60) Provisional application No. 63/330,595, filed on Apr. 13, 2022.

(51) Int. Cl. A61F 5/44

(52) U.S. Cl. CPC A61F 5/4404

(57) ABSTRACT

Various implementations include a urinary catheter drainage bag coupling device that includes a clip configured to removably secure a urinary catheter drainage bag to a surface. The clip includes a first arm and a second arm. The first arm has a first proximal portion and a first distal portion spaced apart from the first proximal portion. The second arm has a second proximal portion and a second distal portion spaced apart from the second proximal portion. The first and second proximal portions are coupled to each other. The first and second distal portions are configured to be biased toward a closed position by a spring force and urgable toward an opened position. In use, the first and second distal ends are closer to each other in the closed position than in the opened position. In various implementations. the device further includes a tether.

### CROSS REFERENCE TO RELATED APPLICATIONS

This PCT application claims priority to, and the benefit of, U.S. Provisional Patent Application No. 63/330,595, filed Apr. 13, 2022, which is incorporated by reference in its entirety.

### BACKGROUND

Between 15% and 25% of patients require an indwelling urinary catheter for their hospital stay. Catheter-associated urinary tract infections (CAUTIs) may be a complication of these devices, causing the highest number of infections (greater than 560,000) compared to other hospital acquired infections. Certain studies have shown that patients with limited mobility can have increased risk of developing CAUTIs. Thus, it is common to help patients mobilize while they have an indwelling catheter, to meet milestones or undergo therapy

Many frontline providers do not have a designated or standard device to hang the drainage bag to decrease backflushing into the bladder. Some tie tourniquets or disposable gloves at the bottom of IV poles to hang the drainage bag, whereas others unable to walk with IV poles, may hang it on a walker or hold the bag while ambulating. This approach can cause a greater instances of catheter displacement, leading to trauma to the urinary track and an increased risk of urinary track infection.

Thus, there is a need for a device to couple a drainage bag to a patient's gown or another object and is flexible, easy to utilize, and safe to help patients and frontline providers consistently and easily keep catheter drainage bags at an appropriate level

### SUMMARY

Disclosed herein are urinary catheter drainage bag coupling devices. In various implementations the device includes a clip having a first arm and a second arm. The first arm has a first proximal portion and a first distal portion spaced apart from the first proximal portion. The second arm has a second proximal portion and a second distal portion spaced apart from the second proximal portion. The first proximal portion and the second proximal portion are coupled to each other, and when in use, the first distal portion and the second distal portion are configured to be biased toward a closed position by a spring force and urgable toward an opened position. The first distal portion and the second distal portion are closer to each other in the closed position than in the opened position.

In various implementations, the first arm and the second arm are resiliently deformable to create the spring force. In various implementations, the clip further includes a spring to create the spring force. In various implementations, the clip further includes a resilient member to create the spring force.

In various implementations, the first arm further includes a first middle portion disposed between the first proximal portion and the first distal portion, wherein the second arm further includes a second middle portion disposed between the second proximal portion and the second distal portion, wherein the first arm and the second arm extend substantially parallel to an arm plane, wherein a projection of the first middle portion and the second middle portion onto the arm plane intersect each other.

In various implementations, the first middle portion extends from a top side of the first proximal portion to a top side of the first distal portion, wherein the second middle portion extends from a bottom side of the second proximal portion to a bottom side of the second distal portion such that the first middle portion and second middle portion are configured to form an interlocking fit.

In various implementations, application of force to the first proximal portion and the second proximal portion causes the clip to move from the closed position to the opened position.

In various implementations, the first distal portion and the second distal portion each include one of more teeth, wherein in use the one or more teeth extend from the first distal portion and the second distal portion toward each other.

In some implementations, the one or more teeth of the first distal portion are interlockable with the one or more teeth of the second distal portion in the closed position.

In some implementations, the one or more teeth have a triangular cross-section.

In some implementations, the first distal portion includes a first tooth portion extending along a first longitudinal axis and a second tooth portion extending along a second longitudinal axis that is perpendicular to the first longitudinal axis, wherein the first longitudinal axis and the second longitudinal axis intersect each other such that the first tooth portion and the second tooth portion at least partially define a first quadrant, a second quadrant, a third quadrant, and a fourth quadrant, wherein the first tooth portion and the second tooth portion each have a width as measured perpendicularly to the first longitudinal axis and the second longitudinal axis respectively, wherein the width of the first tooth portion and the width of the second tooth portion taper from a largest width to a smallest width as the first tooth portion and the second tooth portion extend away from the first distal portion.

In some implementations, the second distal portion includes a first tooth, a second tooth, a third tooth, and a fourth tooth, wherein the first tooth is disposable in the first quadrant, the second tooth is disposable in the second quadrant, the third tooth is disposable in the third quadrant, and the fourth tooth is disposable in the fourth quadrant such that the first tooth, the second tooth, the third tooth, and the fourth tooth each include tapered surfaces that correspond to the tapers of the first tooth portion and the second tooth portion.

In some implementations, the one or more teeth includes one or more rows of angled ridges.

In some implementations, the first distal portion and the second distal portion each include a frictional surface.

In some implementations, the clip comprises a polymer.

In some implementations, the device further comprises a tether having a first tether portion and a second tether portion spaced apart from the first tether portion, wherein the first tether portion is coupled to the clip

In some implementations, the clip defines a clip opening and the first tether portion is disposed within the clip opening. In some implementations, the second tether portion defines a barbed portion. In some implementations, the barbed portion includes a plurality of barbs spaced along a longitudinal axis of the tether. In some implementations, the clip opening is a first clip opening, the clip further defining a second clip opening, wherein the barbed portion is disposable within the second clip opening.

In some implementations, the tether further includes a middle tether portion disposed between the first tether portion and the second tether portion, wherein the middle tether portion defines at least one tether openings, wherein the second tether portion is disposable within one of the at least one tether openings.

In some implementations, the tether comprises a resilient material. In some implementations, the tether comprises a polymer (e.g., a flexible polymer).

In some implementations, the clip further includes one or more hooks coupled to the clip. In some implementations, at least one of the one or more hooks is disposed at a proximal end of the clip. In some implementations, the one or more hooks includes a plurality of hooks.

Various other implementations include a device including: a clip having a first arm and a second arm. The first arm has a first proximal portion and a first distal portion spaced apart from the first proximal portion. The second arm has a second proximal portion and a second distal portion spaced apart from the second proximal portion. The first distal portion has a first surface and the second distal portion includes a second surface. The first proximal portion and the second proximal portion are coupled to each other. The first arm is further configured to be interlockably engaged with the second arm such that when the first arm and second arm are engaged, the first surface and the second surface face towards each other. When in use, the first distal portion and the second distal portion are configured to be biased toward a closed position by a spring force and urgable toward an opened position. The first distal portion and the second distal portion are closer to each other in the closed position than in the opened position.

These and other objects and features of the present invention will become more fully apparent from the following description and appended claims, or may be learned by the practice of the invention as set forth hereinafter.

### BRIEF DESCRIPTION OF THE DRAWINGS

Example features and implementations are disclosed in the accompanying drawings. However, the present disclosure is not limited to the precise arrangements and instrumentalities shown.

FIG. 1A shows a perspective view of a urinary catheter drainage bag coupling device, according to one implementation.

FIG. 1B shows a perspective view of the urinary catheter drainage bag coupling device of FIG. 1A with the tether coupled to the clip.

FIG. 1C shows a top view of the urinary catheter drainage bag coupling device of FIG. 1A.

FIG. 2 shows a detailed perspective view of the first set of teeth and the second set of teeth of the urinary catheter drainage bag coupling device of FIG. 1A.

FIG. 3 shows a perspective view of a urinary catheter drainage bag coupling device, according to another implementation.

FIG. 4A shows a perspective view of a urinary catheter drainage bag coupling device, according to another implementation.

FIG. 4B shows a top view of the urinary catheter drainage bag coupling device

of FIG. 4A.

FIG. 4C shows a top view of the clip of the urinary catheter drainage bag coupling device of FIG. 4A and 4B.

FIG. 4D shows a top view of the tether of the urinary catheter drainage bag coupling device of FIG. 4A and 4B.

FIG. 5A shows a perspective view of a urinary catheter drainage bag coupling device, according to another implementation.

FIG. 5B shows a top view of the urinary catheter drainage bag coupling device of FIG. 5A.

FIG. 6A shows a perspective view of a urinary catheter drainage bag coupling device, according to another implementation.

FIG. 6B shows a top view of the urinary catheter drainage bag coupling device of FIG. 6A.

FIG. 7 depicts a urinary catheter drainage bag coupling device according to one implementation being used to secure a catheter drainage bag to an IV pole.

FIG. 8 depicts a urinary catheter drainage bag coupling device according to one implementation being used to secure a catheter drainage bag to a patient's gown.

### DETAILED DESCRIPTION

The devices, systems, and methods disclosed herein provide for coupling a drainage bag to a gown or other object with a device that is flexible, easy to utilize, and safe to help patients and frontline providers consistently and easily keep catheter drainage bags at an appropriate level. The device can be disposable so that there is no risk of contamination between patients. The device can also be soft, in case a patient were to inadvertently bump or fall into it, there would be no or little injury. The device can be universally used on IV poles, walkers, or attach to a patient's gown.

Various implementations include a urinary catheter drainage bag coupling device. In some implementations, the device includes a clip and a tether. The clip includes a first arm and a second arm. The first arm has a first proximal portion and a first distal portion spaced apart from the first proximal portion. The second arm has a second proximal portion and a second distal portion spaced apart from the second proximal portion. The first proximal portion and the second proximal portion are coupled to each other. The first distal portion and the second distal portion are configured to be biased toward a closed position by a spring force and urgable toward an opened position. The first distal portion and the second distal portion are closer to each other in the closed position than in the opened position. The tether has a first tether portion and a second tether portion spaced apart from the first tether portion. The first tether portion is coupled to the clip.

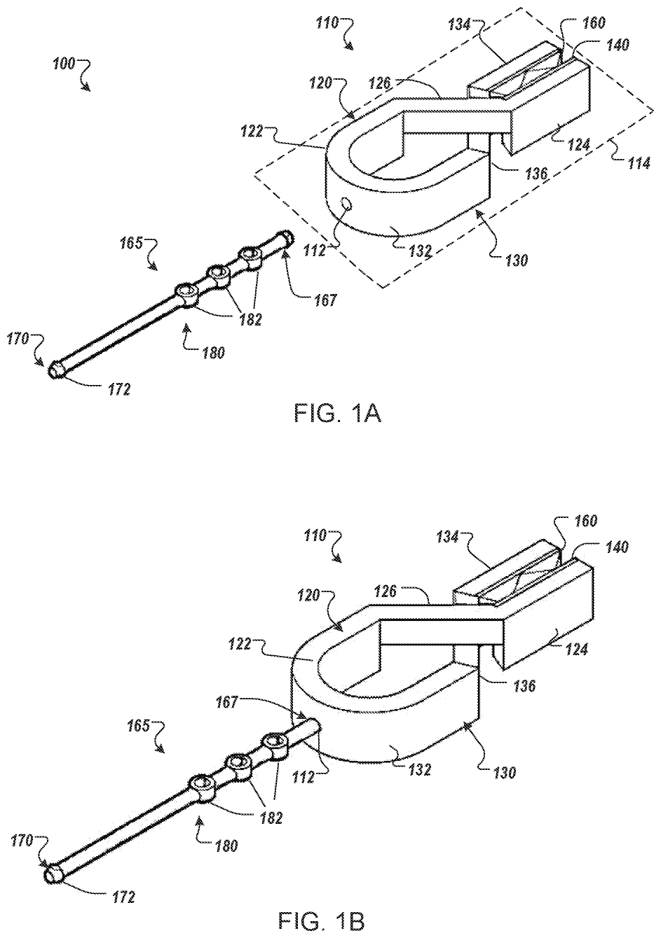

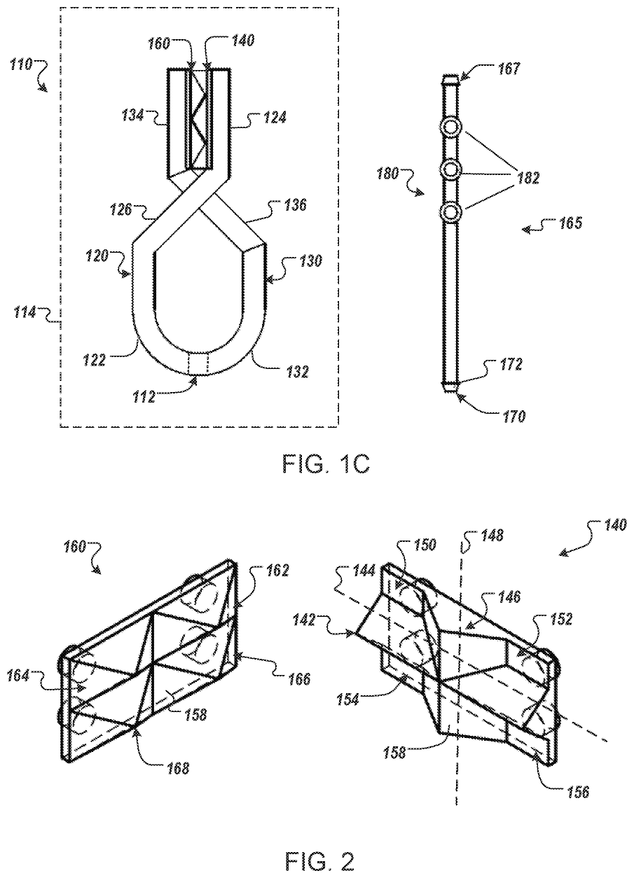

FIGS. 1A-2 show a urinary catheter drainage bag coupling device 100, according to various aspects of one implementation. The device 100 includes a clip 110 and a tether 165.

The clip 110 includes a first arm 120, a second arm 130, and an arm plane 114. The first arm 120 has a first proximal portion 122, a first distal portion 124 spaced apart from the first proximal portion 122, and a first middle portion 126 disposed between the first proximal portion 122 and the first distal portion 124. The second arm 130 has a second proximal portion 132, a second distal portion 134 spaced apart from the second proximal portion 132, and a second middle portion 136 disposed between the second proximal portion 132 and the second distal portion 134.

The first proximal portion 122 and the second proximal portion 132 are coupled to each other. The clip 110 further defines a clip opening 112 adjacent the first proximal portion 122 and the second proximal portion 132. The first arm 120 and the second arm 130 extend substantially parallel to the arm plane 114 such that a projection of the first middle portion 126 and the second middle portion 136 onto the arm plane 114 intersect each other. The intersection of the projections of the first middle portion 126 and the second middle portion 136 form a partial “figure-eight” shape of the clip 110.

The first arm 120 and the second arm 130 are made of a resiliently deformable material. The first distal portion 124 and the second distal portion 134 are biased toward a closed position by a spring force created by the resiliently deformable first arm 120 and second arm 130. The first distal portion 124 and the second distal portion 134 are urgable toward an opened position. The first distal portion 124 and the second distal portion 134 are closer to each other in the closed position than in the opened position. In use, force can be applied to the first proximal portion 122 and the second proximal portion 132 to cause the clip 110 to move from the closed position to the opened position. The clip 110 can be moved from the open position to the closed position to clamp the first distal portion 124 and the second distal portion 134 onto an object to couple the device 100 to the object.

Although the clip 110 shown in FIGS. 1A-2 is made of resiliently deformable material to create the spring force for biasing the clip 110 toward the closed position, in other implementations, the clip includes a spring to create the spring force. In some implementations, the clip includes a resilient member to create the spring force.

The term “resilient member” as used herein means an object that has the ability to return to an original form after the shape of the object has been changed, for example, by being bent, compressed, or stretched. A resilient member may be made from any material that exhibits this property.

As seen in FIGS. 1A-2, the first distal portion 124 of the first arm 120 includes a first set of teeth 140, and the second distal portion 134 of the second arm 130 includes a second set of teeth 160. The first set of teeth 140 extend from an inner surface of the first distal portion 124 toward the second distal portion 134, and the second set of teeth 160 extend from an inner surface of the second distal portion 134 toward the first distal portion 124.

As seen in FIG. 2, each of the teeth of the first set of teeth 140 and the second set of teeth 160 have a triangular cross-section.

The first set of teeth 140 shown in FIG. 2 includes a first tooth portion 142 extending along a first longitudinal axis 144 and a second tooth portion 146 extending along a second longitudinal axis 148 that is perpendicular to the first longitudinal axis 144. The first longitudinal axis 144 and the second longitudinal axis 148 intersect each other such that the first tooth portion 142 and the second tooth portion 146 at least partially define a first quadrant 150, a second quadrant 152, a third quadrant 154, and a fourth quadrant 156. The first tooth portion 142 and the second tooth portion 146 each have a width as measured perpendicularly to the first longitudinal axis 144 and the second longitudinal axis 148 respectively The width of the first tooth portion 142 and the width of the second tooth portion 146 taper from a largest width to a smallest width as the first tooth portion 142 and the second tooth portion 146 extend away from the first distal portion 124 of the first arm 120.

The second set of teeth 160 shown in FIG. 2 includes a first tooth 162, a second tooth 164, a third tooth 166, and a fourth tooth 168. The first tooth 162 is disposable in the first quadrant 150, the second tooth 164 is disposable in the second quadrant 152, the third tooth 166 is disposable in the third quadrant 154, and the fourth tooth 168 is disposable in the fourth quadrant 156 such that the first tooth 162, the second tooth 164, the third tooth 166, and the fourth tooth 168 each include tapered surfaces that correspond to the tapers of the first tooth portion 142 and the second tooth portion 146.

The shape of the first set of teeth 140 and the second set of teeth 160 are configured such that the first set of teeth 140 and the second set of teeth 160 contact each other and are interlockable with each other when the clip 110 is in the closed position.

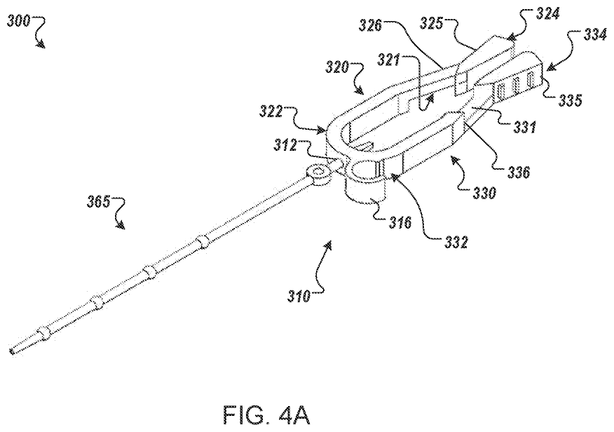

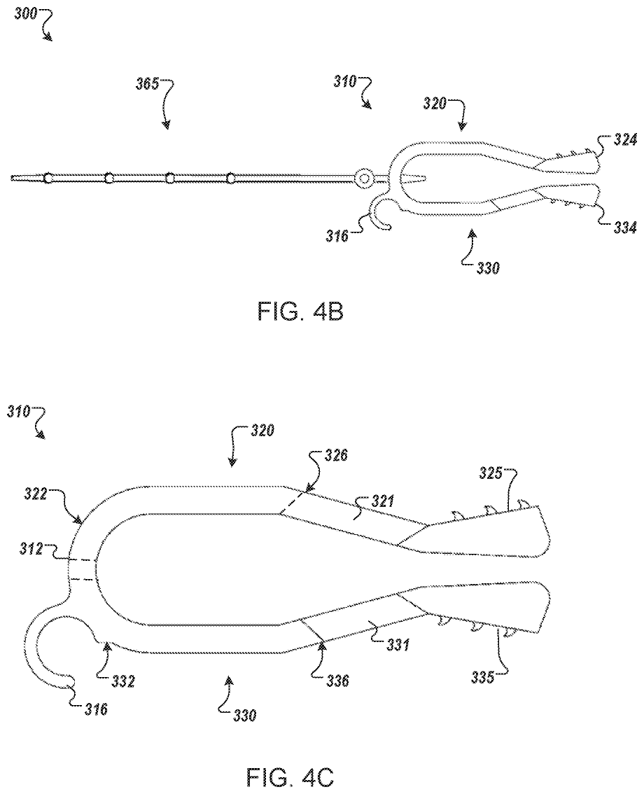

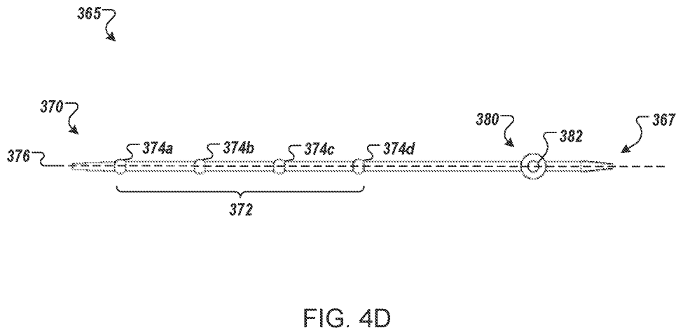

Although the teeth of the first set of teeth 140 and the second set of teeth 160 have a triangular cross-sectional shape, in other implementations, one or more of the teeth have any other cross-sectional shape. For example, the clip 410 shown in FIGS. 4A-SB include a first set of teeth 440 and a second set of teeth 460 each arranged in rows of angled ridges. The angled ridges of the first and second sets of teeth 440, 460 can provide increased gripping strength to firmly grasp an object (e.g., a urinary catheter drainage bag). Although the first set of teeth 140, 440 and the second set of teeth 160, 460 shown in FIGS. 1A-2 and 5A-5B have a particular configuration to increase the holding force of the teeth when they are interlocked in the closed position, in other implementations, the first set of teeth and the second set of teeth can have any configuration to increase the holding strength, to prevent damage to the object to which they are coupled, for ease of manufacturing, for reduced costs, etc. In some implementations, the clip includes any number of teeth or no teeth. In some implementations, the teeth are integrally formed with the clip, which can decrease the manufacturing complexity and therefore reduce production costs. However, in other implementations, the clip and teeth are formed as two or more separate pieces which can later be joined into a single unitary structure before use using conventional assembly techniques (e.g., chemical adhesives, mechanical fasteners, and/or thermal adhesion). In this regard, different materials offering different structural properties can be utilized to enhance properties such as gripping strength of the device.

The first and second sets of teeth 140, 160 of the clip 110 shown in FIGS. 1A-2 further include a frictional surface 158 to increase the holding strength of the clip 110, but in other implementations, the teeth or any other portion of the clip can include any other coating, material, or feature to increase the holding strength of the clip.

The tether 165 has a first tether portion 167, a second tether portion 170 spaced apart from the first tether portion 167, and a middle tether portion 180 disposed between the first tether portion 167 and the second tether portion 170. The first tether portion 167 is disposed within the clip opening 112 to couple the first tether portion 167 to the clip 110.

The second tether portion 170 defines a barbed portion 172, and the middle tether portion 180 defines three tether openings 182, wherein the second tether portion 170 is disposable within one of the tether openings 182. The tether 165 is made of a deformable material and can be deformed such that the barbed portion 172 of the second tether portion 170 can be inserted through one of the three tether openings 182 to form a loop. The barbed portion 172 is easily insertable through the tether opening 182 but couples the second tether portion 170 to the middle tether portion 180 once inserted. The loop can be used to couple the device 100 to another object. Thus, the clip 110 can be coupled to one of a catheter drainage bag and another object while the tether 165 can be coupled to the other of the catheter drainage bag and the other object, effectively coupling the catheter drainage bag to the other object.

Although the middle portion 180 of the tether 165 shown in FIGS. 1A-2 includes three tether openings 182, in other implementations, the middle tether portion, or any other portion of the tether, can include any number of tether openings or no openings.

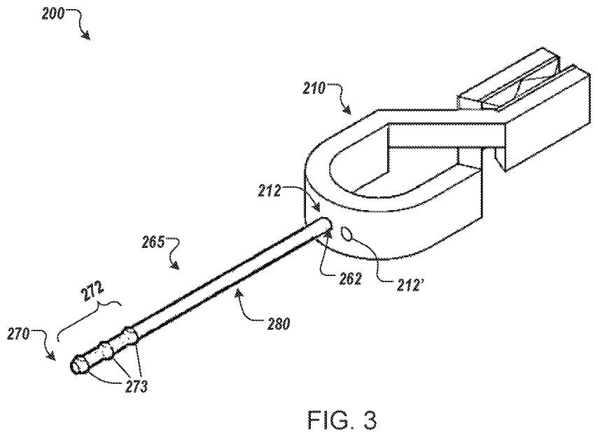

FIG. 3 shows another device 200, according to another implementation. In this implementation, the clip opening 212 is a first clip opening 212, and the clip 210 further defines a second clip opening 212′. The first tether portion 262 of the tether 265 is disposed within and coupled to the first clip opening 212, as with the device 100 of FIGS. 1A-2. In this implementation, the middle tether portion 280 does not define any tether openings, and the barbed portion 272 of the second tether portion 270 includes a plurality of barbs 273 axially spaced along a longitudinal axis of the tether 265. The second tether portion 270 is insertable into the second clip opening 212′ such that one of the plurality of barbs 273 is coupled to the second clip opening 212′ to form a loop. The second tether portion 270 can be further inserted into the second clip opening 212′ to reduce the circumference of the loop based on the size of the object to which the tether 265 is being coupled. Although the tether 265 shows a barbed portion 272 having three barbs 273, in other implementations, the tether can include any number of barbs or none at all. In some implementations, the second tether portion is retained in the second clip opening by another means such as by a frictional force.

FIG. 4A-4D show a urinary catheter drainage bag coupling device 300, according to another implementation. The device 300 includes a clip 310 and a tether 365.

The clip 310 includes a first arm 320 and a second arm 330. The first arm 320 has a first proximal portion 322 and a first distal portion 324 spaced apart from the first proximal portion 322. The second arm 330 has a second proximal portion 332 and a second distal portion 334 spaced apart from the second proximal portion 332. The first distal portion 324 includes a first surface 325 extending from an outer side of the first arm and the second distal portion 334 includes a second surface 335 extending from an outer side of the second arm 330. The first proximal portion 322 and the second proximal portion 332 are coupled to each other.

In various implementations, the first arm and second arm of the clip can be formed in an unclasped arrangement. Although the clip depicted in FIGS. 4A-4C is shown in an unclasped arrangement, the first arm 320 is configured to be interlockably engaged with the second arm 330 such that when the first arm 320 and second arm 330 are engaged, the first surface 325 and the second surface 335 face towards each other. The first arm 320 and second arm 330 can be resiliently deformed such that a first middle portion 326 of the first arm 320 overlays a second middle portion 336 of the second arm 330. While in use, the first distal portion 324 and the second distal portion 334 are configured to be biased toward a closed position by a spring force and urgable toward an opened position. Thus, the first distal portion 324 and the second distal portion 334 are closer to each other in the closed position than in the opened position. Forming the clip of a device in an unclasped arrangement can advantageously increase the gripping strength between the first and second distal portion caused by the spring force and lower the production cost.

The first arm 320 shown in FIGS. 4A-4C further includes a first middle portion 326 disposed between the first proximal portion 322 and the first distal portion 324. The second arm 330 further includes a second middle portion 336 disposed between the second proximal portion 332 and the second distal portion 334. The first arm 320 includes a first groove 321 extending along a portion of the first middle portion 326. The second arm 330 includes a second groove 331 extending along a portion of the second middle portion 336. The first groove 321 and second groove 331 are complementary to each other such that when the first arm 320 and second arm 330 are engaged, the first groove 321 and second groove 331 are coupled to form an interlocking fit. The clip 310 further comprises a resilient material such that the interlocking fit of the first arm 320 and second arm 330 creates a tension urging the first surface 325 of the first distal portion 324 towards the second surface 335 of the second distal portion 334 urging the first surface and second surface towards each other.

In various implementations, the device 300, when the first arm 320 and second arm 330 are interlockably engaged, creates a spring force that allows the distal end (i.e., the first distal portion 324 and second distal portion 334) to grip an object (e.g., a urinary catheter drainage bag). Preferably, the spring force is sufficiently large to firmly grip the object without slippage, while still being readily deformable by a user. In various implementations, the spring force is caused by a tension between the interlockably engaged first arm 320 and second arm 330 such that the first arm 320 and second arm 330 urge the first surface 325 of the first distal portion 324 towards the second surface 335 of the second distal portion 334. In various implementations, a user can apply a force (e.g., by squeezing the first proximal portion 322 and second proximal portion 332 towards each other) such that the first distal portion 324 and second distal portion 334 are moved from a closed position to an open position. When the device 300 is interlockably engaged, the first surface 325 and second surface 335 are closer together in the closed position than in the open position.

As shown in FIG. 4A-4B and 4D, the urinary catheter drainage bag coupling device 300 can further include a tether 365 and a clip opening 312. The tether 365 has a first tether portion 367, a second tether portion 370 spaced apart from the first tether portion 367, and a middle tether portion 380 disposed between the first tether portion 367 and the second tether portion 370. In use, the first tether portion 367 can be disposed within the clip opening 312 to couple the first tether portion 367 to the clip 310. In some implementations, the first tether portion includes a flanged end which can be removably inserted into the clip opening. Once inserted into the clip opening to couple the tether to the clip, the flanged end obstructs the separation of the first tether portion from the clip.

The second tether portion 370 defines a barbed portion 372, and the middle tether portion 380 defines a tether opening 382, wherein the second tether portion 370 is disposable within one of the tether opening 382. The tether 365 is made of a deformable material and can be deformed such that the barbed portion 372 of the second tether portion 370 can be inserted through the tether opening 382 to form a loop. The barbed portion 372 is easily insertable through the tether opening 382 but couples the second tether portion 370 to the middle tether portion 380 once inserted. The loop can be used to couple the device 300 to another object (e.g., an IV pole). Thus, the clip 310 can be coupled to one of a catheter drainage bag and another object while the tether 365 can be coupled to the other of the catheter drainage bag and the other object, effectively coupling the catheter drainage bag to the other object. The barbed portion 372 of FIG. 4D includes four rounded barbs 373 (373a, 373b, 373c, 373d) spaced along a longitudinal axis 376 of the tether 365. However, in other implementations, additional or fewer barbs can be included depending on the particular dimensions of the object which the tether couples with.

The urinary catheter drainage bag coupling device 300 further includes a hook 316. The book 316 is coupled to the second proximal portion 332 of the clip 310 where it can be used to organize tubing or wires of the patient. The hook 316 is disposed on the second proximal portion 332 and substantially coplanar with the first and second arms 320, 330 of the clip 310. However, in other implementations, the hook can be positioned and/or oriented elsewhere on the clip.

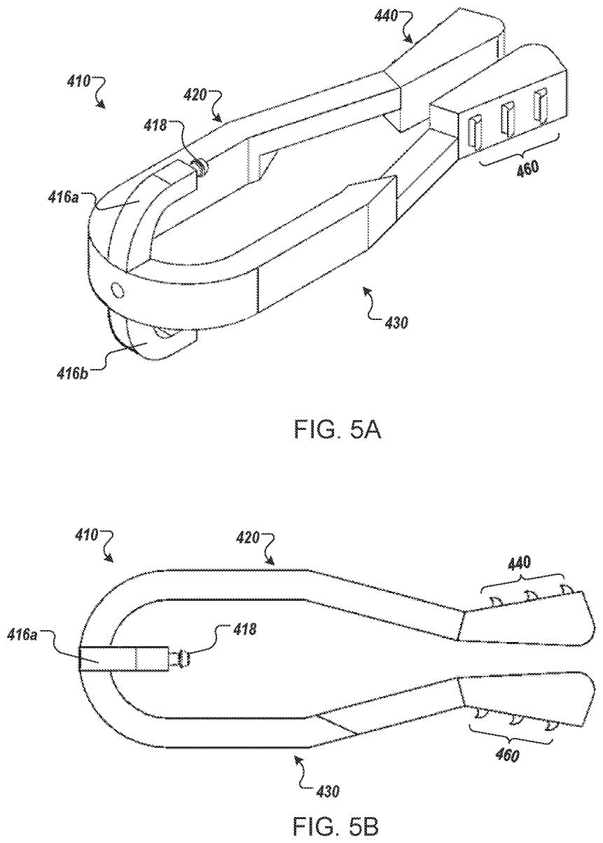

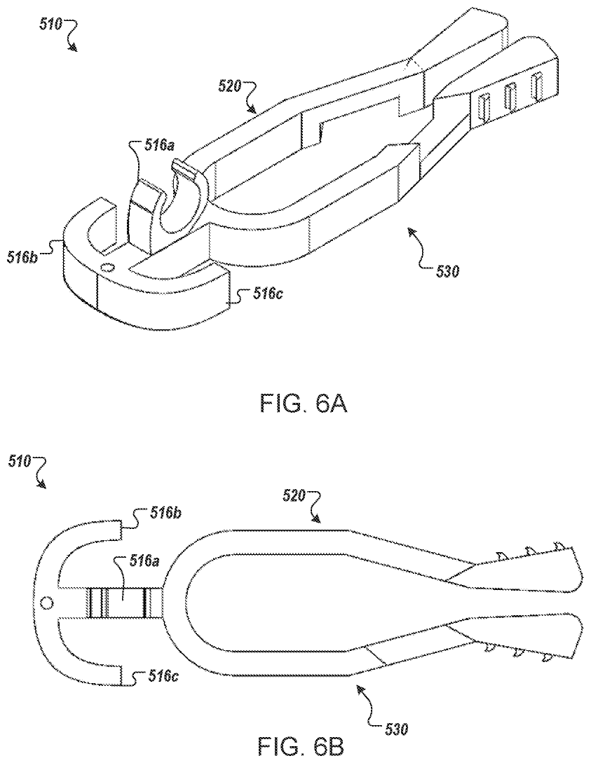

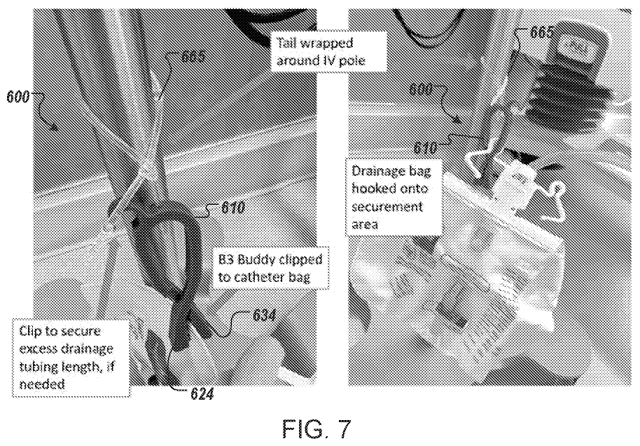

Although FIGS. 4A-4C illustrates a clip 310 having a single hook 316, various other implementations include clips having a plurality of hooks (e.g., 2 or more, 3 or more, 4 or more). For example, FIGS. 5A-5B each show a clip 410 of a urinary catheter drainage bag coupling devices with an alternative arrangement of a first hook 416a and second hook 416b. First hook 416a and second hook 416b are positioned opposite one another and extend perpendicularly from the first arm 420 and second arm 430 of the clip 410. In some implementations, the one or more hooks can further include a flexible strap removably disposed proximate to an opening to prevent tubes from shifting. For example, the first hook 416a and second hook 416b each include a protrusion 418. A flexible strap (e.g., a rubber band) can extend from the protrusion 418 on the first hook 416a to the protrusion 418 on the second hook 416b, effectively securing tubing to the clip 410. Referring now to FIGS. 6A-6B, the clip 510 includes three hooks 516a, 516b, 516c disposed along the proximal end of the clip 510. The clip 510 includes a first hooks 516a and second hook 516b which are both coplanar to the first arm 520 and second arm 530 of the clip 510. The clip 510 further includes a third hook 516c oriented normal to the first hook 516a and second hook 516b. In various implementations, the clip includes one or more hooks having a complementary size to receive one or more tubes associated with a catheter drainage bag.

The clips 110, 210, 310, 410, and 510 and the tethers 165, 265, 365 shown in FIGS. 1A-6B are made of a polymer, but in other implementations, one or both of the clip and tether are made of any other material. In various implementations, the clip comprises a polymer (e.g., a thermoplastic). Examples of suitable polymeric materials for the clip include, for example, polyurethane, acrylonitrile butadiene styrene (ABS), polyether ether ketone (PEEK), nylon, polyimide, polyethylene terephthalate (PET), polyethylene terephthalate glycol (PETG), and/or the like. In various implementations, the tether comprises a flexible polymeric material securely loop around an object. For example, the flexible polymeric material can include polypropylene, polyethylene, polyethylene terephthalate, polyethylene terephthalate (PET), polyester, plasticized polyvinyl chloride (PPVC), polycarbonate, and copolymers thereof. In various implementations, one or both of the clip and tether are made from a disposable polymeric material. However, a polymeric material (e.g., a molded polyethylene, polyvinyl chloride, polystyrene, or the like) that is either reusable and/or sterilizable is also contemplated.

The clips and tethers disclosed herein can be fabricated together or separately using any suitable technique known in the art. By way of example, the presently disclosed clip and/or tether can be produced using 3D printing, injection molding, additive manufacturing, compression molding, thermoforming, or a combinations thereof. In various implementations, the clip is manufactured as a single piece. However, it is also contemplated that the clip can include separate pieces that are later joined.

FIG. 7 shows another device 600 according to another implementation to secure a catheter drainage bag. The device 600 includes a clip 610 and a flexible tether 665. The tether 665 is fastenably wrapped around an IV pole to secure the clip 610 at a fixed height on the pole via a frictional force. The tether 665 is further configured such that the effective diameter of the loop can be adjusted to accommodate various sizes and shapes of poles and bars to couple with. The clip 610 is securably coupled both to the tether 665 and the catheter drainage bag. The drainage bag can be placed between the first and second distal portions 624, 634 of the clip 610 as shown in the left panel of FIG. 7, or can be secured to another location on the clip 610 as depicted in the right panel.

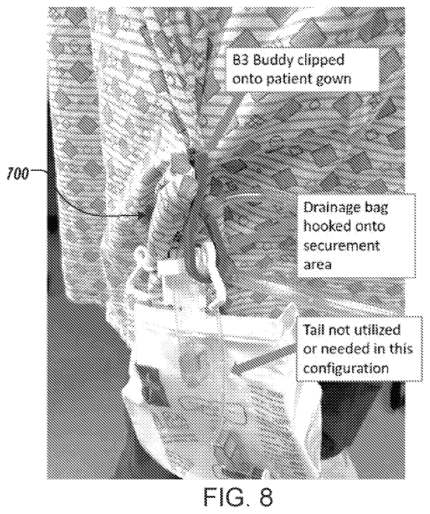

Although each of the devices 100, 200, 300, 600 depicted in FIGS. 1A-1B, 3, 4A-4B, and 7 are shown to include both a clip 110, 210, 310, 610 and tether 165, 265, 365, 665, some implementations of the device include a clip without the tether. For example, FIG. 8 shows a device 700 according to the present disclosure coupling a drainage bag directly to a patient's gown without using a tether.

A number of example implementations are provided herein. However, it is understood that various modifications can be made without departing from the spirit and scope of the disclosure herein. As used in the specification, and in the appended claims, the singular forms “a,” “an,” “the” include plural referents unless the context clearly dictates otherwise. The term “comprising” and variations thereof as used herein is used synonymously with the term “including” and variations thereof and are open, non-limiting terms. Although the terms “comprising” and “including” have been used herein to describe various implementations, the terms “consisting essentially of” and “consisting of” can be used in place of “comprising” and “including” to provide for more specific implementations and are also disclosed.

Disclosed are materials, systems, devices, methods, compositions, and components that can be used for, can be used in conjunction with, can be used in preparation for, or are products of the disclosed methods, systems, and devices. These and other components are disclosed herein, and it is understood that when combinations, subsets, interactions, groups, etc. of these components are disclosed that while specific reference of each various individual and collective combinations and permutations of these components may not be explicitly disclosed, each is specifically contemplated and described herein. For example, if a device is disclosed and discussed each and every combination and permutation of the device are disclosed herein, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed systems or devices. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

What is claimed is:

1. A urinary catheter drainage bag coupling device, the device comprising:
a clip including a first arm and a second arm, the first arm having a first proximal portion and a first distal portion spaced apart from the first proximal portion, the second arm having a second proximal portion and a second distal portion spaced apart from the second proximal portion, wherein the first proximal portion and the second proximal portion are coupled to each other, wherein in use the first distal portion and the second distal portion are configured to be biased toward a closed position by a spring force and urgable toward an opened position, wherein the first distal portion and the second distal portion are closer to each other in the closed position than in the opened position.

2. The device of claim 1, wherein the first arm and the second arm are resiliently deformable to create the spring force.

3. The device of any of claims 1-2, wherein the clip further includes a spring to create the spring force.

4. The device of any of claims 1-3, wherein the clip further includes a resilient member to create the spring force.

5. The device of any of claims 1-4, wherein the first arm further includes a first middle portion disposed between the first proximal portion and the first distal portion, wherein the second arm further includes a second middle portion disposed between the second proximal portion and the second distal portion, wherein the first arm and the second arm extend substantially parallel to an arm plane, wherein a projection of the first middle portion and the second middle portion onto the arm plane intersect each other.

6. The device of claim 5, wherein the first middle portion extends from a top side of the first proximal portion to a top side of the first distal portion, wherein the second middle portion extends from a bottom side of the second proximal portion to a bottom side of the second distal portion such that the first middle portion and second middle portion are configured to form an interlocking fit.

7. The device of any of claims 1-6, wherein application of force to the first proximal portion and the second proximal portion causes the clip to move from the closed position to the opened position.

8. The device of any of claims 1-7, wherein the first distal portion and the second distal portion each include one of more teeth, wherein in use the one or more teeth extend from the first distal portion and the second distal portion toward each other.

9. The device of claim 8, wherein the one or more teeth of the first distal portion are interlockable with the one or more teeth of the second distal portion in the closed position.

10. The device of any of claims 8-9, wherein the one or more teeth have a triangular cross-section.

11. The device of any of claims 8-10, wherein the first distal portion includes a first tooth portion extending along a first longitudinal axis and a second tooth portion extending along a second longitudinal axis that is perpendicular to the first longitudinal axis, wherein the first longitudinal axis and the second longitudinal axis intersect each other such that the first tooth portion and the second tooth portion at least partially define a first quadrant, a second quadrant, a third quadrant, and a fourth quadrant, wherein the first tooth portion and the second tooth portion each have a width as measured perpendicularly to the first longitudinal axis and the second longitudinal axis respectively, wherein the width of the first tooth portion and the width of the second tooth portion taper from a largest width to a smallest width as the first tooth portion and the second tooth portion extend away from the first distal portion.

12. The device of claim 11, wherein the second distal portion includes a first tooth, a second tooth, a third tooth, and a fourth tooth, wherein the first tooth is disposable in the first quadrant, the second tooth is disposable in the second quadrant, the third tooth is disposable in the third quadrant, and the fourth tooth is disposable in the fourth quadrant such that the first tooth, the second tooth, the third tooth, and the fourth tooth each include tapered surfaces that correspond to the tapers of the first tooth portion and the second tooth portion.

13. The device of any of claims 8-10, wherein the one or more teeth includes one or more rows of angled ridges.

14. The device of any of claims 1-13, wherein the first distal portion and the second distal portion each include a frictional surface.

15. The device of any of claims 1-14, wherein the clip comprises a polymer.

16. The device of any of claims 1-15 further comprising a tether having a first tether portion and a second tether portion spaced apart from the first tether portion, wherein the first tether portion is coupled to the clip.

17. The device of claim 16, wherein the clip defines a clip opening and the first tether portion is disposed within the clip opening.

18. The device of any of claims 16-17, wherein the second tether portion defines a barbed portion.

19. The device of any of claims 16-18, wherein the tether further includes a middle tether portion disposed between the first tether portion and the second tether portion, wherein the middle tether portion defines at least one tether openings, wherein the second tether portion is disposable within one of the at least one tether openings.

20. The device of any of claims 18-19, wherein the clip opening is a first clip opening, the clip further defining a second clip opening, wherein the barbed portion is disposable within the second clip opening.

21. The device of any of claims 18-20, wherein the barbed portion includes a plurality of barbs spaced along a longitudinal axis of the tether.

22. The device of any of claims 16-21, wherein the tether comprises a resilient material.

23. The device of any of claims 16-22, wherein the tether comprises a polymer.

24. The device of any of claims 1-23 wherein the clip further includes one or more hooks coupled to the clip.

25. The device of claim 24, wherein at least one of the one or more hooks is disposed at a proximal end of the clip.

26. The device of any of claims 24-25, wherein the one or more hooks includes a plurality of hooks.

27. A urinary catheter drainage bag coupling device, the device comprising:
a clip including a first arm and a second arm, the first arm having a first proximal portion and a first distal portion spaced apart from the first proximal portion, the second arm having a second proximal portion and a second distal portion spaced apart from the second proximal portion, wherein the first distal portion includes a first surface and the second distal portion includes a second surface, wherein the first proximal portion and the second proximal portion are coupled to each other, wherein the first arm is configured to be interlockably engaged with the second arm such that when the first arm and second arm are engaged, the first surface and the second surface face towards each other, wherein in use the first distal portion and the second distal portion are configured to be biased toward a closed position by a spring force and urgable toward an opened position, wherein the first distal portion and the second distal portion are closer to each other in the closed position than in the opened position.
